(19) United States
(12) Patent Application Publication
(10) Pub. No.: US 2022/0218590 A1
(43) Pub. Date: Jul. 14, 2022

(54) COMPOSITIONS AND METHODS FOR REMOVAL OF SKIN ADHESIVE PATCHES, PRODUCTS, AND RESIDUAL ADHESIVES

(71) Applicant: ABEND FRITZ VENTURES LLC, East Aurora, NY (US)

(72) Inventors: Michael FRITZ, East Aurora, NY (US); Sarah ABEND-FRITZ, East Aurora, NY (US)

(21) Appl. No.: 17/611,123

(22) PCT Filed: May 12, 2020
(86) PCT No.: PCT/US2020/032516

§ 371 Date: Nov. 12, 2021

Related U.S. Application Data
(60) Provisional application No. 62/847,927, filed on May 14, 2019.

(51) Int. Cl. A61K 8/92; A61Q 19/10; A61K 8/67; B65D 47/18

(52) U.S. Cl. CPC A61K 8/922; A61Q 19/10; A61K 2800/5922; B65D 47/18; A61K 2800/87; A61K 8/678

(57) ABSTRACT

Provided are compositions, methods and articles of manufacture, for use in removing an adhered substrate from skin of an individual. The composition comprise a mixture of oils that include Jojoba oil, Tee Tree Oil, Sweet Orange Oil, Peppermint Oil, Vitamin E oil. The methods involve application of the composition onto a substrate adhered on the skin of an individual, allowing the composition to loosen the adhesive, and removing the substrate from the skin of the individual. Containers that include the compositions are includes, as are articles of manufacture that include the containers, and may include packaging with a printed indication of the ingredients of the composition, and instructions for using the composition.

### CROSS-REFERENCE TO RELATED APPLICATIONS

This application claims priority to U.S. provisional patent application No. 62/847,927, filed May 14, 2019, the disclosure of which is incorporated herein by reference.

### FIELD OF INVENTION

The present invention relates to the removal of adhesive patches, appliances, residues, and products that are secured to the skin.

### BACKGROUND

The removal of adhesive products designed to temporarily adhere to a user's skin for a variety of purposes can cause significant discomfort, pain, and damage to the skin when they are removed from the body.

The types of damage to the skin that a user might experience are: Dryness, irritation, inflammation, cracking, tearing, bruising, bleeding, and infection.

Prior to the present disclosure, available products that claim to remove adhesive products and residues from the skin contain chemicals and compounds with documented health warnings including cancer risks and environmental hazards, as well as petroleum and alcohol based ingredients. In addition, these adhesive removal products require adhesive patches and bandages to be pried and pulled from the skin, and do not adequately penetrate the fibers of the adhered product. The combination of pulling from the skin and the chemicals contained in these products exacerbate the trauma to the skin and pose additional health risks. Thus, there is an ongoing and unmet need for a product that is free of alcohol, chemicals, and fossil fuel derived ingredients, for safely dissolving the bond of adhesive products while still on the skin, and prior to removal. The present disclosure is pertinent to this need.

### SUMMARY

The present disclosure provides compositions, methods and kits for use in adhesive substrate removal from skin. The compositions comprise a mixture of botanically based liquids, which allows a user to remove adhesives and adhesive secured products from the body without causing pain or damage to the skin. The methods involve use of the compositions to remove substrates that are adhered to a user's skin. The disclosure includes contains that contain a described composition, and may also include an applicator.

In embodiments, the disclosure provides mixtures of oils that, in addition to providing an alternative to previously available adhesive removal agents, benefit the skin with antimicrobial and other beneficial properties as further described herein. The compositions comprise a mixture of oils that include Jojoba Oil, Tea Tree oil, Sweet Orange Oil, Peppermint Oil, and Vitamin E Oil. Containers that contain the described compositions are included, as are articles of manufacture that include the containers, packaging, and may include printed material the describes the components of the composition, and instructions for using the composition.

In non-limiting and illustrative embodiments, the disclosure provides compositions, devices and methods which relate to removal one or more adhered patches from the torso of an individual. In this regard, FIG. 1 provides a depiction of the torso 100 of an individual with two adhesive patches 110 adhered to the skin of the torso 100 of the individual. The adhesive patches 110 are connected to one another by medical tubing 120 as a non-limiting representation of insulin infusion set.

FIG. 2 provides a depiction of container containing a composition of the disclosure in use. The container 210 includes a removable applicator 200 in the form of a dropper cap. The container 210 contains a mixture of oils 220.

FIG. 3 provides a depiction of using the applicator 200 to dispense the mixture 220 onto one of the two adhered patches 110 so that the patch absorbs the mixture 220, illustrated by the migration of the composition 220 from the application 200 to the patch 110.

FIG. 4 provides a depiction of two patches 110 on the torso 100 of the individual. Both patches 110 are shown as having absorbed the mixture of oils 220. Thus, the patches 110 are allowed to stay on the individual for a period of time while the oil 220 is absorbed by the patches 110.

FIG. 5 provides a depiction of loosening one of the two the patches 110 that has absorbed the mixture 220 after a period of time. FIG. 6 shown removal a loosened patch 110 after the patch 110 has been exposed to the mixture 220.

FIG. 7 provides depiction of a removed patch 110a from the skin. Patch 110 that is still adhered to skin of the torso 100 may be removed in the same manner.

### BRIEF DESCRIPTION OF THE DRAWINGS

Embodiments of the disclosure are illustrated by the following figures, which are not intended to be limiting.

FIG. 1 provides a depiction of the torso 100 of an individual with two adhesive patches 110 adhered to the skin of the torso 100 of the individual. The adhesive patches 110 are connected to one another by medical tubing 120. The adhesive patches 110 and medical tubing 120 are a non-limiting representation of insulin infusion set.

FIG. 2 provides a depiction of a device containing a composition of the disclosure in use. The device includes an applicator 200 and a container 210. The container contains a mixture of oils 220.

FIG. 3 provides a depiction of using the applicator 200 to dispense the mixture 220 onto one of the two adhered patches 110 so that the patch absorbs the mixture 220.

FIG. 4 provides a depiction of both patches 110 on the torso 100 of the individual having absorbed the mixture of oils 220.

FIG. 5 provides a depiction of loosening one of the two the patches 110 after the period of time described as in FIG. 4, that patch having absorbed the mixture 220.

FIG. 6 provides depiction of removal a loosened patch 110 after contacting the patch 110 with the mixture 220.

FIG. 7 provides depiction of a removed patch 110a from the skin. Patch 110 that is still adhered to skin of the torso 100 may be removed in the same manner.

### DETAILED DESCRIPTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Every numerical range given throughout this specification includes its upper and lower values, as well as every narrower numerical range that falls within it, as if such narrower numerical ranges were all expressly written herein.

Description of the materials and process steps are described further below. The disclosure includes the proviso that any single or combination of reagents and steps may be excluded from the claims of this application or patent. In embodiments, any composition described herein may comprise, consist essentially of, or consist of the described components.

The present disclosure was developed in part based on a need for the described compositions and methods based on a diagnosis of a child of the present inventors with Type 1 diabetes at a young age. As part of management of his condition, use of an insulin pump and continuous glucose monitor (CGM) became necessary. In order to secure the patches and insulin pump leads, it was necessary to adhere them to our child's body with strong adhesive patches designed to remain on the skin for several days. Removing these devices produced a great deal of pain, which was exacerbated by sensitive skin. Further, his skin was damaged by removal of the patches almost immediately. In addition to the significant pain and discomfort that the removal of these powerful adhesives caused, the skin became very irritated. As a consequence, skin infections developed at the sites of the adhered devices, and required antibiotic treatments. Additionally, removal of the devices caused raised skin, cracking, dryness, tearing, and bleeding.

In order to alleviate these effects caused by removal of the devices, we used commercially available removing agents. However, these agents contained ingredients that, instead of improving the skin condition and alleviating the described damage, only caused more damage to the skin. The ingredients included components as alcohol, and in certain cases included warnings on labels and safety data sheets, advising of health risks associated with use of the products, including but not limited to a risk of developing cancer.

In view of these drawbacks of existing products, and after significant experimentation over a period of months, we developed the presently described compositions and methods as an alternative to the unacceptable and dangerous products that were previously available for removal of substrates adhered to the skin. These compositions comprise a mixture of essential oils that readily dissolve the adhesive patches of the CGM and insulin pump leads without pain or irritation, while providing beneficial oils and botanicals used in cosmetic skin care products to help protect and condition the skin.

In embodiments, a composition of the disclosure comprises a mixture of Jojoba, such as a carrier oil, with Tea Tree oil, Sweet Orange Oil, Peppermint Oil, and Vitamin E oil. The combination of these ingredients when blended in a proper ratio has been highly advantageous in the removal of the adhesive patches and residual adhesive from the skin of our child. Moreover, his skin has been free of infection and irritation since the initial time the described composition was used to remove the adhesive devices.

In embodiments, the disclosure provides a mixture of oils. In embodiments, the oils are selected from Jojoba, Tea Tree, Sweet Orange, Peppermint, Vitamin E, and all combinations thereof. In embodiments, a composition of this disclosure consists essentially of a combination of all of the foregoing oils. In embodiments, a composition of the disclosure consists of a combination of all of the foregoing oils.

Each of the oils used in the compositions and methods of the disclosure is known in the art and is commercially available. In embodiments, the oils have the following physical characteristics:

TABLE 1

| Ingredient | Specific Gravity | lbs./US Gallon | lbs./ounce |
|---|---|---|---|
| JOJOBA | 0.863 | 7.21 | 0.056 |
| TEA TREE | 0.894 | 7.46 | 0.058 |
| SWEET ORANGE | 0.842 | 7.03 | 0.055 |
| PEPPERMINT | 0.896 | 7.48 | 0.058 |
| VITAMIN E | 0.95 | 7.93 | 0.062 |

In embodiments, a composition of the disclosure comprises the following amounts of each component in one a total of one fluid ounce (oz.):

TABLE 2
Volume of each ingredient in total volume of 1 fluid oz.

| | |
|---|---|
| JOJOBA | 0.49 oz. |
| TEA TREE | 0.34 oz. |
| SWEET ORANGE | 0.10 oz. |
| PEPPERMINT | 0.05 oz. |
| VITAMIN E | 0.02 oz. |

TABLE 3
Ratio of Ingredients

| Ingredient | Ratio | % |
|---|---|---|
| JOJOBA | 10 | 48.78% |
| TEA TREE | 7 | 34.15% |
| SWEET ORANGE | 2 | 9.76% |
| PEPPERMINT | 1 | 4.88% |
| VITAMIN E | 0.5 | 2.44% |

The composition can be provided in any volume. Using the relative amounts described in the foregoing Tables, the relative amounts of each ingredient can be present in a volume of from 0.1 ounce, to one or more gallons.

In embodiments, using Table 2 as an example, a gallon of the composition comprises 128 ounces, wherein each ounce has the relative amounts for each ounce, as shown in Table 2.

In embodiments, the composition has a ratio of components. In embodiments, taking Table 3 as an example, Jojoba is may be considered a carrier oil and may comprise approximately half of the total volume of the composition (e.g., approximately 48.78%, as reflected in the 0.49 oz. of a total volume of 1 oz., as in Table 1). If Jojoba is assigned a value of 10 as in Table 3, the Jojoba is used in a 10:7 ratio relative to Tea Tree oil (e.g., 0.7×48.78%=34.15% for Tea Tree oil). Likewise, the Sweet Orange is present in a 2:1 ratio relative to the Peppermint oil (e.g., the Peppermint oil is one half the Sweet Orange, e.g., 4.88% is half of the 0.76% of the Sweet Orange). In the same manner, Peppermint oil is present in a 1:0.5 ratio to the Vitamin E (e.g., 0.5×4.88% is 2.44%). Thus, in one embodiments, a composition of the disclosure is prepared in the following manner: The oils are measured in volume according to the following ratios:
Jojoba (10): Tea Tree (7): Sweet Orange (2): Peppermint (1): Vitamin E (0.5).

Stated differently, for every one fluid ounce, the composition contains approximately 0.49 oz. Jojoba oil, approximately 0.34 oz. of Tee Tree oil, approximately 0.10 oz. of Sweet Orange oil, approximately 0.05 oz. of Peppermint oil, and approximately 0.02 oz. of Vitamin E oil. In embodiments, the oils are prepared in a sterile manner, using any suitable mixture devices, appliances, and the like.

Each of the described oils is known in the art, and is commercially available. For example, each of the described oils is available from New Directions Aromatics Inc™ (Ontario, Canada), accessible at www.newdirectionsaromatics.com/products/essential-oils/. The description of each oil as it exists on the New Directions Aromatics Inc. webpage is incorporated herein by reference, as each said description exists on the effective filing date of this application or patent.

In particular, as is known to those skilled in the art, Jojoba oil is the liquid produced in the seed of the Simmondsia chinensis. It contains Palmitic acid, Palmitoleic acid, Stearic acid, Oleic acid, Arachidic acid, 11-Eicosenoic acid, Behenic acid, Erucic acid, Lignoceric acid, and Nervonic acid, in mole fractions that are known in the art. In embodiments, the Jojoba oil comprises a clear organic carrier oil.

As also known in the art, Tea Tree oil is an oil derived from the leaves of the tea tree, Melaleuca alternifolia. Tea tree oil is defined by the International Standard ISO 4730 (“Oil of Melaleuca, terpinen-4-ol type”), containing terpinen-4-ol, γ-terpinene, and α-terpinene as about 70% to 90% of whole oil, while p-cymene, terpinolene, α-terpineol, and α-pinene collectively account for some 15% of the oil. In embodiments, Tea Tree Oil comprises a minimum of 30% Terpinen-4-ol.

As is known in the art, Orange Oil, referred to herein as Sweet Orange oil, is an essential oil produced by cells within the rind of orange fruit (Citrus sinensis). It contains Limonene, α-Pinene, Sabinene and β-Pinene, Myrcene, Octanal, Linalool, δ-3-Carene, and Decanal. In embodiments, the Sweet Orange oil comprises approximately 95.35% d-Limonene.

As is known in the art, Vitamin E oil comprises tocopherol. In embodiments, cosmetic grade Vitamin E oil is used. In embodiments, the Vitamin E oil comprises α-Tocopheryl acetate. In embodiments, the Vitamin E oil comprises 67%-87% D-Alpha-Tocopherol and the remainder comprising another organic oil, such as soybean oil/

As is known in the art, Peppermint Oil comprises an extract cross between water mint and spearmint. It comprises were menthol and menthone, and may contain (±)-menthyl acetate, 1,8-cineole, limonene, beta-pinene and beta-caryophyllene.

Thus, any oil described herein can be recognized as having the above-described components.

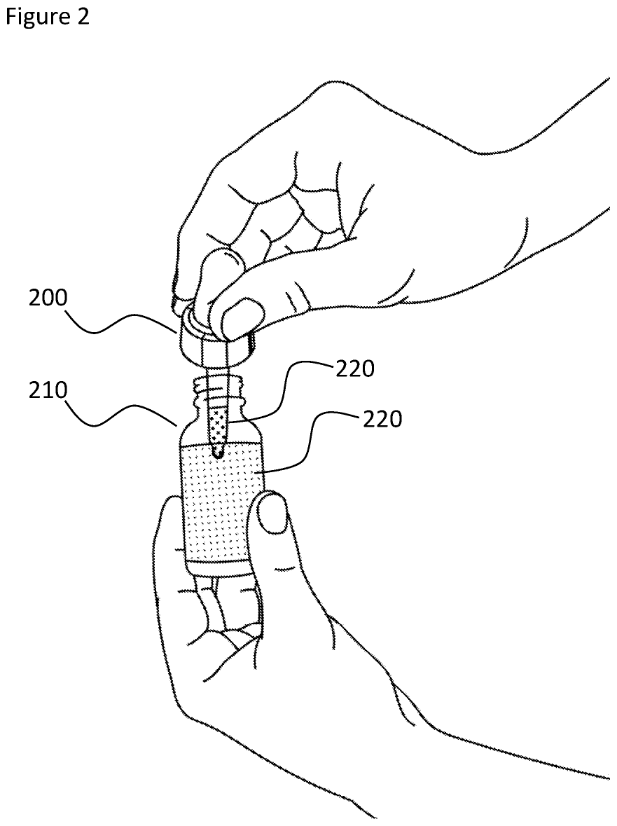

In embodiments, any suitable container can be used to contain the oil mixture. In embodiments, the container comprises a glass or plastic container. In embodiments, the container comprises a bottle, a jar, an ampule, or a vial. In embodiments, the container is transparent. In embodiments, the container is colored, and or is opaque. In embodiments, the container is a glass jar, as generally depicted by the container (210) of FIG. 2.

In embodiments, the container has a liquid capacity of from 1 oz., to 128 oz., including all numbers there between, and all ranges of numbers the between. In embodiments, the container may comprise a larger liquid volume, and thus may be provided as a barrel, or drum, for example, for distribution to distributors who may fill smaller containers for a variety of downstream consumers.

In embodiments, the container comprises a removable top. In embodiments, the top comprises a screw cap. In embodiments, the cap comprises a detachable cover which covers an opening to access the liquid composition, wherein the detachable cover may be connected to the top via any suitable tether. In embodiments, an initial seal between the cap and the contents of the container, such as a removable foil seal, may be provided.

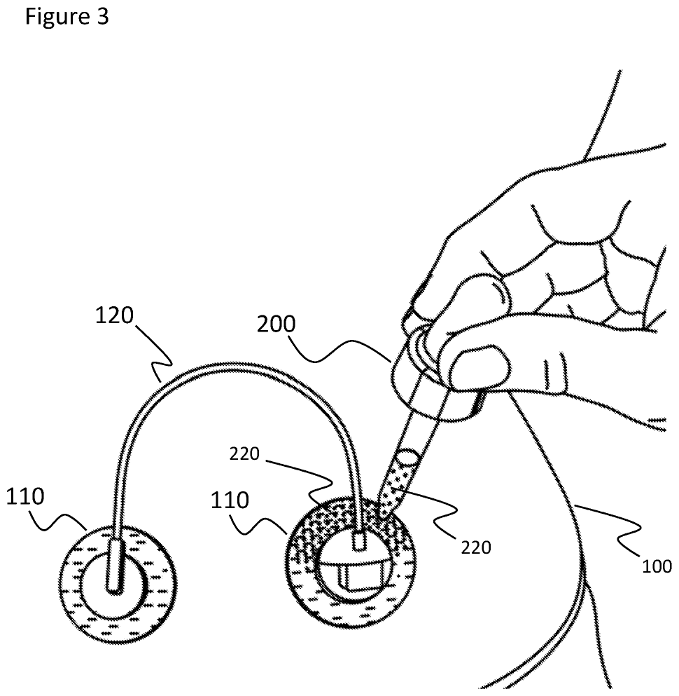

In embodiments, a composition of the disclosure is provided with a container and any suitable dispenser, such as any type of applicator. In embodiments, the applicator is integrated into a removable top. In embodiments, the applicator is selected from a dropper, which comprises, for example, a glass or plastic tube, and a squeezable bulb formed of rubber or any other suitable flexible material, and thus may be provided as a dropper-cap assembly. A non-limiting embodiment of a dropper cap assembly that is uses as an applicator is shown in FIGS. 2 and 3. In particular, FIG. 2 (200) shows a dropper cap applicator as used to draw a composition 220 into it from container 210. In alternative embodiments, the applicator comprises a syringe. In embodiments, the applicator comprises a glass or plastic pipette, which may include a disposable pipette.

In embodiments, the disclosure provides an article of manufacture comprising a composition of the disclosure. The article of manufacture may comprise packaging that contains the container. The container, or the packaging, or both, may comprise printed material, such as a label and/or an insert, or printed information on the packaging itself, that provides an indication of the contents of the container, and may further comprise instructions for using the composition to remove any adhered substrate from the skin of a user.

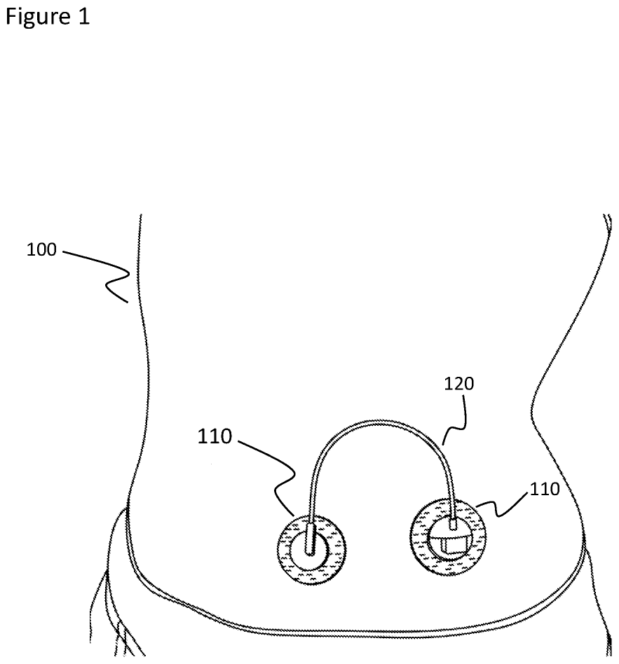

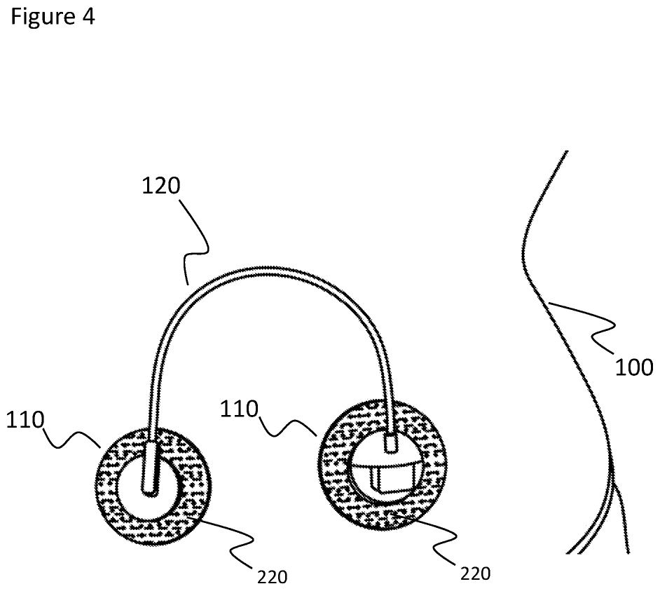

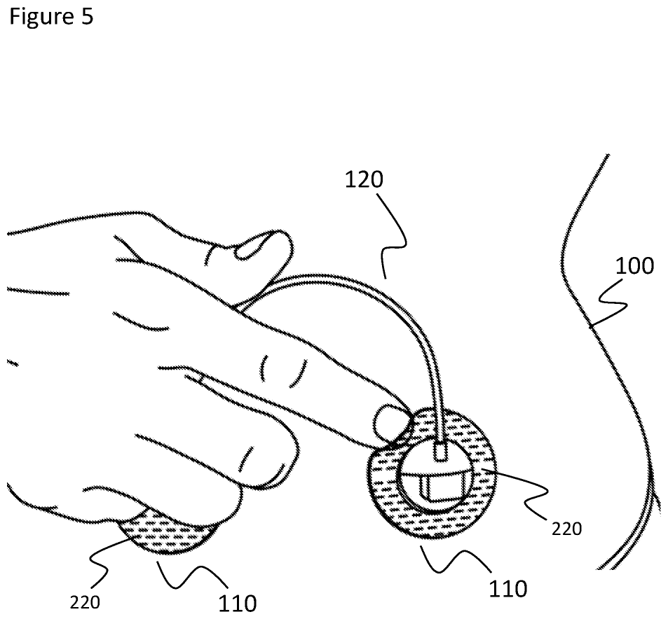

In embodiments, the compositions and devices of this disclosure are suitable for removal of any substrate that is adhered to the skin of an individual. The type of substrate is not particularly limited. In embodiments, the substrate is a porous material, and may therefore comprise any type of, for example, a medicated patch, including but not limited to a transdermal drug delivery patch. In embodiments, the patch is generally circular in shape, but other shapes are not excluded from the disclosure. In non-limiting embodiments, the adhered substrate has an adhered surface area of 1 to 15 cm². In embodiments, one or more patches or other adhered substrates may be removed from skin of an individual. The adhered substrate may be positioned on any location of the skin of the individual, including but not limited to an arm, leg, torso, neck, and buttocks. In an embodiment, the substrate is adhered to a torso of the individual. As a non-limiting example, two patches 110 are depicted as adhered to a torso 100 of an individual in, for example, at least FIGS. 1, 3, and 5. The patches or other adhered substrates may be connected to other components, such as tubing. A non-limiting example of tubing 120 is shown FIGS. 1 and 3-7, which represent the tubing of an insulin fusion set.

In embodiments, the adhered device comprises a backing, which may or may not be fully or partially porous. In embodiments, the device adhered to the skin of the user comprises a membrane. In embodiments, the device adhered to the skin of the user comprises a matrix filler. In embodiments, device is selected from a single layer drug-in adhesive, a multi-layer drug in adhesive, or it may comprise a drug reservoir. In embodiments, the adhesive device comprises a matrix, such as a solid or semi-solid matrix. In embodiments, the adhesive device comprises polyethylene, a polyester, a paper, a cotton or other textile including any woven fabric, a non-woven fabric, or rayon and/or rayon acetate.

In embodiments, the adhesive device comprise a vapor patch. In embodiments, the adhesive device to be removed comprises an adhesive tape, including but not limited to athletic tape. In embodiments, the adhesive comprises a glue. In embodiments, the adhesive comprises an acrylic or an acrylate compound. In embodiments, the adhesive comprises a silicone compound. In embodiments, the adhesive comprises a pressure sensitive acrylate adhesive. In embodiments, the adhesive comprises latex. In embodiments, the adhesive comprises an epoxy. In embodiments, the adhesive comprises a natural or synthetic resin.

In embodiments, the adhesive device that is removed using the compositions and methods of the disclosure comprises a medical device or a medical device component. In embodiments, the adhesive device is a component of a medical pump, including but not necessarily limited to an insulin pump, a dialysis pump, and/or a monitor, such as a glucose monitor, a hear monitor, and the like. In embodiments, the adhesive is a component of a partially implantable medical device. In embodiments, the adhesive is a component of an electrode. In embodiments, the adhesive is a component of a dressing, including but not necessarily limited to a bandage.

In embodiments, the oils comprise a homogenous mixture, which may comprise a solution. In embodiments, the compositions of this disclosure are alcohol free. In embodiments, the compositions of this disclosure are acetone free. In embodiment, the compositions of the disclosure are petroleum free.

In an embodiment, the disclosure provides a method of making the composition. The method generally comprises measuring the oils in relative amounts as described above, and combining them sequentially, or concurrently in, for example, any suitable mixing container, such as a sterile glass container, and mixing, blending, or otherwise agitating the combined oils so that they, for example, form a homogenous mixture. The composition is then, for example, bottled in any appropriate sized container, such as amber glass bottles.

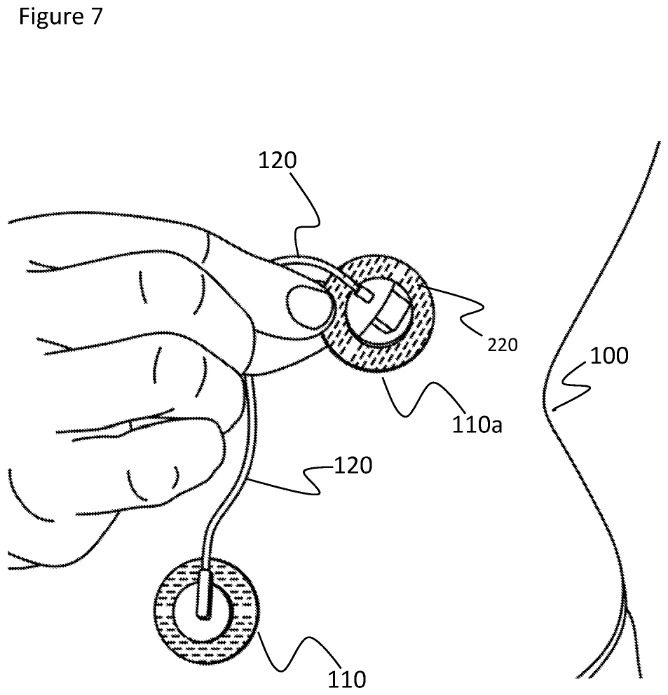

In an embodiment, the disclosure comprises removing an adhered substrate from the skin of an individual. In an embodiment, the method comprises contacting an adhered substrate with a composition of this disclosure and removing the adhered substrate. The composition may also be dispensed onto residual adhesive on the skin to facilitate removal of the residual adhesive, and thereafter removing the residual adhesive. In embodiments, removal of the adhered substrate is performed as generally shown in FIGS. 3-6. A removed patch 110a is shown in FIG. 7.

In an embodiment, a method of the disclosure comprises dispensing a suitable volume of a composition of the disclosure onto, and/or beneath, an adhered substrate, allowing a period of time for the composition to be absorbed by the substrate, and/or or to migrate from a perimeter of the substrate to some or all of the area between the substrate and the skin, to thereby loosen the adhesive-skin bonding. In embodiments, the adhesive is dissolved. In embodiments, the composition disrupts adsorption of the adhesive to the skin, and thereby weakens electrostatic forces between the adhesive and the skin. In embodiments, the volume of the composition dispensed varies by the area of the adhered substrate, and/or by the location of the adhesive on the substrate. In embodiments, a volume of from 0.1 to 5 ounces is dispensed. In embodiments, approximately 1 milliliter is used.

In embodiments, the composition is allowed to remain on, in, and/or beneath the substrate for a period of approximately 30 seconds to five minutes. In embodiments, the composition is allowed to remain in contact with the substrate for about three minutes. In embodiments, the substrate is mechanically manipulated, such as by hand, to improve penetration and/or other access of the composition to the adhesive.

After a suitable amount of time, the method of the disclosure comprises removing the substrate from the skin. The substrate may be removed by any suitable application of force, such as peeling. The method further comprises removing residual adhesive after removal of the substrate, such as by either wiping the skin using the retained composition, or dispending additional composition onto the skin to facilitate residual adhesive removal with, for example, additional wiping.

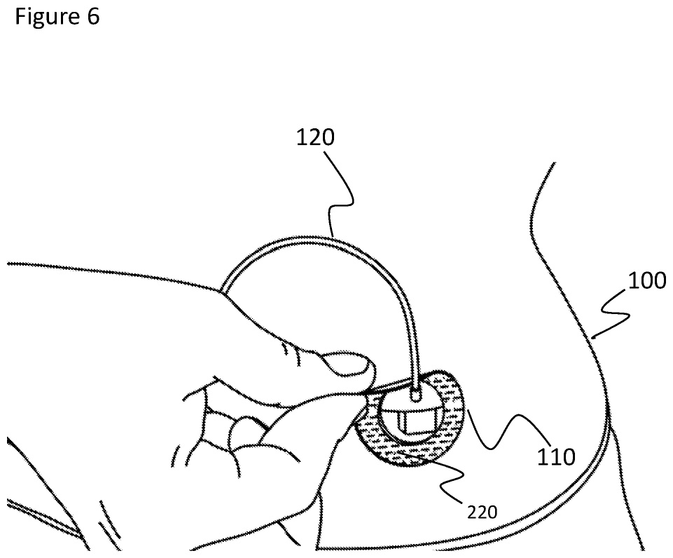

As discussed above, a non-limiting embodiment of the disclosure is demonstrated by way of the following description and figures. This example is illustrated by performing a method of the disclosure on an insulin pump tubing secure to the outside of a an adhesive fabric patch 100 as depicted in FIG. 1. Using the integrated cap/dropper (FIG. 2, 200) the user squeezes, for example, a rubber cap that is a component of the applicator 200 and upon release, draws the described composition 220 up into the representative dispenser 200 shown as a glass dropper 200. The user then squeezes a sufficient amount of the described composition 220 out of the dropper on to the fabric support of the adhesive on the patch 110 (FIG. 3) or the residual adhesive on the skin until the fibers of the adhesive support have been at least partially, or fully saturated. The user then waits for an adequate period of time, such as for 3 minutes so that the adhesive patches 110 have absorbed the mixture 220. In this time period, and without intending to be bound by any particular theory, it is considered that the composition dissolves the adhesive. The user then removes the adhesive product 110 by gently lifting it from the skin (FIGS. 6 and 7), and may wipe residual adhesive from the skin using any suitable material, such as a clean fabric cloth or tissue. Additional cleaning of the site is not necessary.

While the disclosure has been described through specific embodiments, routine modifications will be apparent to those skilled in the art and such modifications are intended to be within the scope of the present disclosure.

1. A method of removing an adhered substrate from skin of an individual, the method comprising contacting a material comprising the adhered substrate with a composition comprising Jojoba oil, Tee Tree Oil, Sweet Orange Oil, Peppermint Oil, Vitamin E Oil, such that the contacting the material comprising the substrate loosens the adhesive, and subsequently removing the substrate from the skin.

2. The method of claim 1, wherein the composition comprises, per fluid ounce, approximately 0.49 ounce of the Jojoba oil, approximately 0.34 ounce of the Tea Tree Oil, approximately 0.10 ounce of the Sweet Orange Oil, approximately 0.05 ounce of the Peppermint oil, and approximately 0.02 ounce of the Vitamin E oil.

3. The method of claim 2, wherein the composition consists of, per fluid ounce, 0.49 ounce of the Jojoba oil, 0.34 ounce of the Tea Tree Oil, 0.10 ounce of the Sweet Orange Oil, 0.05 ounce of the Peppermint oil, and 0.02 ounce of the Vitamin E oil.

4. The method of claim 1, wherein the composition is applied to the adhered substrate for a period of approximately 30 seconds to three minutes, and wherein optionally the composition is applied a portion of the adhered substrate that is not in contact with the skin of the individual.

5. The method of claim 4, wherein the adhered substrate is comprised by an adhesive patch.

6. A composition comprising a mixture of oils for use in removing an adhered substrate, the composition comprising a mixture of Jojoba oil, Tee Tree Oil, Sweet Orange Oil, Peppermint Oil, and Vitamin E oil.

7. The composition of claim 6, wherein the composition consists essentially of the Jojoba oil, Tee Tree Oil, Sweet Orange Oil, Peppermint Oil, and Vitamin E oil.

8. The composition of claim 7, wherein the composition comprises, per fluid ounce, approximately 0.49 ounce of the Jojoba oil, approximately 0.34 ounce of the Tea Tree Oil, approximately 0.10 ounce of the Sweet Orange Oil, approximately 0.05 ounce of the Peppermint oil, and approximately 0.02 ounce of the Vitamin E oil.

9. A container comprising the composition of claim 7, the container comprising a removable top.

10. The container of claim 9, wherein the container comprises a dispenser.

11. The container of claim 10, wherein the dispenser comprises a dropper.

12. The container of claim 11, wherein the dispenser is integrated into a removable top that is configured to close the container.

13. An article of manufacture, the article comprising a container of claim 9, the article of manufacture further comprising packaging and/or printed material, wherein the printed material provides an indication that the composition in the container is for use in removing an adhered substrate from skin of an individual.

14. The article of manufacture of claim 13, wherein the printed material is provided on a label on the container.

15. The article of manufacture of claim 13, wherein the printed material provides instructions for using the composition in the container for removal of the adhered substrate.

16. A method for preparing a composition suitable for removal of an adhered substrate from skin, the method comprising mixing Jojoba oil, Tee Tree Oil, Sweet Orange Oil, Peppermint Oil, Vitamin E Oil, to thereby form the mixture.

17. The method of claim 16, wherein each fluid ounce of the composition is prepared by mixing together 0.49 ounce of the Jojoba oil, 0.34 ounce of the Tea Tree Oil, 0.10 ounce of the Sweet Orange Oil, 0.05 ounce of the Peppermint oil, and 0.02 ounce of the Vitamin E oil.
